(19) United States
(12) Patent Application Publication
(10) Pub. No.: US 2014/0100542 A1
(43) Pub. Date: Apr. 10, 2014

(54) WOUND CARE ARTICLE WITH AN ENVELOPE THAT IS DEFORMABLE IN A SPECIFIC MANNER

(71) Applicant: Birgit Riesinger, Munster (DE)

(72) Inventor: Birgit Riesinger, Munster (DE)

(21) Appl. No.: 14/044,994

(22) Filed: Oct. 3, 2013

Related U.S. Application Data
(63) Continuation of application No. PCT/EP2012/056170, filed on Apr. 4, 2012.

(30) Foreign Application Priority Data
Apr. 6, 2011 (DE) 10 2011 832.8

(51) Int. Cl. A61F 13/45

(52) U.S. Cl. CPC A61F 13/45
USPC 604/369; 604/385.01

(57) ABSTRACT

The subject matter of the invention is a wound care device having at least one absorbent body which is surrounded by an outer sheath. The sheath comprises means which are configured and/or selected in such a manner that the sheath is at least partially deformable in a target-oriented manner by a volume increase of the absorbent body caused by fluid absorption (FIG. 5a).

### CROSS REFERENCE TO PRIOR APPLICATIONS

The present invention is a continuation and claims priority under 35 U.S.C. §120 and §365(c) to PCT International Patent Application PCT/EP2012/056170, filed Apr. 4, 2012, which claims priority to German Patent Application No. 10 2011 832.8, filed Apr. 6, 2011, all of which are herein incorporated by reference in their entireties.

The invention relates to a wound care device having at least one absorbent body and an outer sheath according to Claim 1.

Such a wound care device is particularly suitable for receiving wound fluid from strongly exudative wounds, particularly chronic wounds, such as those that occur in diabetes, crural ulcer, and similar diseases, for example.

Flat wound dressings have been known for a long time. Flat wound dressings containing superabsorbent' substances, particularly polymers, are also known. Such wound dressings can be a wound dressing for absorbing wound exudates, as described in WO03094813, WO2007051599 and WO0152780 of the applicant of the present invention, for example, to the entire disclosure content of which reference is made here.

In order to accelerate the healing process, it can be of crucial importance whether the wound dressing exerts a certain pressure on the wound, due to the increase in the absorption of wound exudate, or, if desired, the wound dressing leads the absorbed exudate away from the wound, resulting in the formation of a small hollow cavity above the surface of the wound.

The aim of the present invention therefore is to provide a wound care device, wherein the volume increase is guided in a predetermined direction.

This aim is achieved by the wound care device according to the main claim. Additional preferred embodiments are represented by the features of the dependent claims.

Accordingly, the present invention discloses a wound care device having at least one absorbent body, which is surrounded by an outer sheath, characterized in that the sheath comprises means which are designed and/or selected in such a manner that the sheath is at least partially deformable in a target-oriented manner by a volume increase of the absorbent body caused by the absorption of fluid.

The term “wound care device” below denotes a wound dressing, preferably a flat wound dressing or a wound care cloth.

In this context, “deformable in a target-oriented manner” means that the outer sheath around the absorbent body guides the volume increase during the absorption process in a certain direction. This can occur both by expansion of the absorbent body in the direction of the wound or away from the wound, that is to say depending on the configuration in the vertical direction. Moreover, an expansion in the horizontal direction, that is to say over the surface of the wound care device, can also be provided. For the direction, the constitution of the sheath and its specific capacity for expansion in connection with the absorbent body located beneath is crucial, wherein the expansion of the sheath can be reversible or irreversible. In principle, such a behavior can be achieved with a unidirectional resilient material, that is to say a material which can be expanded in one direction but not in the direction orthogonal to the former direction. Such materials are known, for example, from WO2005087159, in which they are used in a joint bandage. In another embodiment, this can be achieved by a skilled combination of resilient and nonresilient materials. Forms of these embodiments are described in further detail in the description.

The special constitution of the sheath becomes clear in reference to the additional variants. Thus, the sheath has a fluid-permeable side turned toward the wound and a side turned away from the wound, wherein at least one of the two sides is resiliently and/or plastically deformable at least in partial zones. It should be understood that the sheath is permeable to fluid at least on the side turned toward the wound. In addition, the sheath forms a barrier against solid secretions, while at the same time, however, allowing the passage of other substances which have exited toward the material section made of absorption material which is arranged within the sheath.

The term “partial zone” means that at least a partial surface of the corresponding side is resiliently and/or plastically deformable. This is of particular relevance if the wound care device is positioned in such a manner that the absorption process is influenced by natural gravity, for example, in the case of a leg dressing in the form of a sleeve. In a preferred embodiment example, the upper section of the sleeve, in the applied state, is resiliently and/or plastically deformable, and the lower section, in this case the portion closer to the foot, is not resilient, so that the lower section can counteract the sagging of the absorbent body which becomes heavier due to the absorption of the wound exudates. In this context, the wound care device according to the invention moreover can have a shape that is adapted to the anatomical circumstances. For this purpose, it can be configured in the shape of a sleeve in such a manner that it can be fitted over an arm, a leg or a joint. In another variant, the wound care device can be adapted to the shape of a heel, an elbow joint, or the like.

In a particular embodiment of the wound care device, an expansion margin is delimited by the sheath around the absorbent body, margin which in terms of its dimensions is adapted to the volume increase of the absorbent body, and which, in the starting state, is 3-90 volume % larger than the absorbent body. Conversely, in a top view of its flat side, the absorbent body has an area (F1), which in the noncrosslinked state is 3-90% smaller than the area (F2) of the flatly placed sheath, and which is freely movable or fixed in the sheath. As a result, additional space is created, in which, particularly at the beginning of the absorption process, the absorbent body can expand in the case of an increase in the volume, before it reaches the resiliently and/or plastically deformable sheath. The expansion margin is thus preferably an expansion margin which is located peripherally around the absorbent body which is in a flat arrangement.

In an additional preferred embodiment of the wound care device according to the invention, at least one lamination layer, preferably a cellulose nonwoven, is arranged between the absorbent body and the sheath. The lamination layer is used to lead the wound exudate taken up by the sheath to the absorbent body and at the same time to prevent a return flow of wound exudate into the wound. The lamination layer is adapted preferably to the inner dimension of the sheath, and accordingly its area is slightly larger than the absorbent body located beneath, so that leaks from the absorbent body can be collected. It is preferable for the lamination layer to be adapted exactly to the expansion margin around the absorbent body. The absorbent body can be covered either only from one side by a lamination layer, or, on the other hand, in a sandwich arrangement from both sides with one or more lamination layers. The lamination layer is preferably made of a cellulose nonwoven without additional superabsorbent polymer components. However, lamination layers with superabsorbent polymers are also possible.

In order to guide the expansion of the absorbent body in a certain direction by expansion which is due to a volume increase caused by the absorption of fluid during the absorption of wound exudate, one side of the sheath can be resiliently and/or plastically deformable. The term “resiliently deformable” denotes a reversible deformation, that is to say the sheath material in this portion has the capacity, after the deformation, of returning to the original state, provided that the so-called stretch limit has not been reached. However, if the stretching exceeds the stretch limit, the material does not return to the original state. The consequence is a permanent deformation due to plastic behavior. Thus, the term “plastically deformable” denotes an irreversible deformation. The degree of deformation depends on the given material of which the sheath is made.

The sheath is preferably made of a hydrophobic material or of a natural material that has been provided with a hydrophobic finishing. The hydrophobic properties of the sheath prevent sticking to the wound surface and they contribute to the capacity of the wound exudate particles to reach the interior of the sheath more rapidly.

In a particular embodiment, the deformable material is a fabric or nonwoven having at least one resilient or plastic component, preferably a chemical fiber, particularly preferably a polyaddition fiber, and most particularly preferably a polyurethane fiber. Straight polyurethane fibers have the property of a high elastic expandability. Polyurethane is available as a fiber under the trade names Elastan, Lycra or Dorlestan and it consists of at least 85% polyurethane. Since, compared to rubber, it can be dyed easily, it is used usually in connection with other fibers for expandable fabrics. The chemical fibers, formally also referred to as synthetic fibers, include, however, not only fibers made of synthetic polymers, but also fibers made of natural polymers, for example, rubber fibers. These fibers made of natural polymers can also be used as elastic components for the sheath in the wound care device according to the invention. In addition, some natural fibers are also suitable, provided that they have a minimum level of resilience or plasticity.

The fabric or nonwoven can consist entirely of the resilient or plastic components, or it can be combined with another material, for example, in combination with other nonexpandable fibers. The sheath consists preferably of a fabric or nonwoven, which has an area-specific weight of at least 20 g/m².

In an alternative embodiment of the wound care device according to the invention, the deformable material is a plastic film. Suitable materials are any types of films having a certain flexibility, so that the film is resiliently or plastically deformable. Suitable plastic films are preferably made of polyethylene or polyurethane. However, in addition, the films can also be made of polystyrene, polyvinyl chloride, polyester or polycarbonate or of synthetic spun silk. In addition, the films can be provided entirely or partially with pores, in particular on the side turned toward the wound, so that the wound exudate reaches the absorbent body via the pores. In a preferred embodiment, the sheath has even perforation in its entirety or partially. The material of the sheath can be structured in such a manner that the sheath has a rough inner surface and a smooth outer surface. It is preferable for the rough inner surface of the sheath to be formed by funnel-shaped perforations, each narrowing in the direction of the inner surface, and draining into a free opening edge (“outward cantilever”). This rough inner face counteracts the shifts of the content of the sheath, so that a securing with glue points can be dispensed with. Accordingly, the smooth outer surface of the sheath material can be formed by arched material sections extending between the perforations. Such a sheath material, in contrast to a sheath material that is flat on both sides, can be referred to as “three dimensional,” and it is known, for example, from DE102006017194 of the applicant of the present invention, to the entire disclosure content of which reference is made here.

To the extent that the absorbent body comprises superabsorbent particles, the pores are adapted to the size of the superabsorbent particles, so that the superabsorbent particles cannot exit from the sheath. The pores or mesh openings of the sheath preferably have a size of 0.05-1.0 mm, and preferably 0.20-0.50 mm. Moreover, it is possible in a preferred manner, to provide that the pores or mesh openings are delimited by the threads or fiber sections, which are approximately arc-shaped in cross section through the sheath, and which point outward with their arched vertices.

In a further embodiment of the wound care device according to the invention, the sheath comprises, on at least one side, a peripheral seam, preferably an ultrasound seam, in order to delimit a deformation of the sheath. It is particularly preferable to provide that the sheath, in a top view onto its flat side, comprises a peripheral overlying part protruding over the seam, and that the absorbent body is free of hard, sharp edges and corners. The sheath is held together by the seam. At the same time, the total extension of the absorbent body is limited or guided in a certain direction. In order to support this effect, the seam can be deformable in some sections resiliently and/or plastically, in a particularly preferred embodiment. As a result, similarly to the textile sector, one achieves that the sheath in the zones with resiliently and/or plastically deformable seams has a high expandability, while in the remaining zones with rigid seams no expansion is possible. Particularly suitable seams are ultrasound seams or welded seams. In another embodiment, the seam is gathered, for example, by using an expandable gathered ribbon strip with a rubber band incorporated therein. As a result, the sheath is easily gathered and it acquires a high expandability at the seams.

In an additional embodiment of the wound care device according to the invention, the deformable sheath has means that are offset by color, which visibly change in the case of a targeted deformation of the sheath by expansion of the absorbent body, and represent an indicator of the extent of the deformation. As a result, the care giving personnel or the patient receives an indication when the absorbent body has reached its maximum extension due to absorption and has to be replaced. For this purpose, various means are available: In a preferred embodiment, the appropriate means is selected from the group of threads, small bands and wires. One or more of these means are visibly incorporated in the sheath and they can tear as a result of the expansion of the absorbent body, so that the number of torn points and/or the distance between two torn ends represent(s) an indication of the volume increase of the absorbent body. In another variant, for example, a color change occurs, in that the incorporated thread loses its color intensity as a result of the stretching during volume increase of the absorbent body, and thus provides an indication of the status of the absorption process. To the extent that a target-oriented deformation of the absorbent body in the direction of the wound is desired, such a means can also be applied to the nonexpandable outer side of the sheath, which faces away from the wound, and it can lead, for example, as a result of the compression of the sheath on the side facing away from the wound, to a visible change of the indicator, in particular due to an increase in color intensity.

The absorbent body is substantially made from a flat absorption material. In addition, the absorbent body can be surrounded by an inner, fluid-permeable sheath. However, this inner sheath does not have to have the same properties with regard to target-oriented deformability as the outer sheath. Common fluid-permeable sheath materials are sufficiently known in connection with the commercial absorption bodies used in the field of wound care.

In an embodiment of the wound care device according to the invention, the absorbent body has several layers, wherein at least one layer is a fabric or nonwoven-like mat. The term “nonwoven” denotes a flat textile structure made of individual fibers, which, in contrast to woven or knitted fabrics, is not made from yarn. Nonwovens retain their structural integrity as a rule due to the adhesion of the individual fibers to each other. They are also referred to as “nonwovens,” and are produced, for example, by fulling the fibers.

The individual layers of the absorbent body preferably have an area-specific weight of at least 300 g/m², wherein the grammage of the proportion of osmotically active substances evenly distributed therein is at least 100 g/m².

In a preferred embodiment, the absorbent body has additional distribution layers. These distribution layers ensure a distribution of the wound exudates over the entire absorbing area of the absorbent body and thus improve the absorption process overall.

In a particularly preferred embodiment of the wound care device according to the invention, the absorbent body has at least partially superabsorbent substances, preferably in the form of particles, granulates, powders, bulk preparations and/or fibers. These superabsorbent substances can also be in the form of compact bodies as a preform, a foam, a fiber knitted fabric, a fiber roving fabric or a fiber nonwoven fabric and/or a fiber wadding or it can be incorporated in such materials. It is particularly preferable to use superabsorbent polymers (SAP). Superabsorbent polymers are plastics capable of taking up by suction a multiple of their own weight—up to 1000 times their own weight—of fluids. Chemically, they are copolymers made of acrylic acid (propenoic acid, C₃H₄O₂) and sodium acrylate (sodium salt of acrylic acid, NaC₃H₃O₂), wherein the ratio of the two monomers to each other can vary. In addition, a so-called core crosslinker (CXL) is added to the monomer solution, which in some sections connects the formed long-chained polymer molecules by chemical bridges to each other (see “crosslinked”). As a result of these bridges, the polymer becomes water insoluble. At the time of the penetration of water or aqueous salt solutions into the polymer particles, the polymer swells and becomes stretched on the molecular plane of this network, so that water can no longer escape unaided.

In a preferred embodiment, the absorbent body is an airlaid. The term “airlaid” denotes a special nonwoven made of cellulose and polyolefin fibers, in which superabsorbent polymers are optionally embedded.

The superabsorbent substances can be incorporated equally into one or more of the layers of the absorbent body, or they can be made completely from them. In particular, it is preferable to provide that the individual layers consist of more than 40 wt % of superabsorbent polymers.

Absorbent bodies of the mentioned type are disclosed, for example, in WO03094813, WO2007051599 and WO0152780 of the applicant of the present invention. The entire disclosure content of the mentioned documents is added to the disclosure content of this document.

In an additional embodiment, the wound care device according to the invention, for the purpose of the attachment to the patient or for the purpose of compressing the wound care device prior to the application on the patient, comprises attachment means that can be connected to each other, preferably hook-and-loop closures and/or adhesive surfaces. The attachment means accordingly can have several functions: for rapid attachment or for rapid application on patients, the wound care devices can comprise rapid closure means, as known, for example, for diapers. Alternatively, however, it is also possible to use bands or adhesive strips. To the extent that the wound space or the edema is to be filled with the wound care device, the attachment means can also be arranged in such a manner that when the means in use, compression or pushing together of the wound care device occurs, so that the wound care device can be adapted optimally to the wound. In addition, such attachment means are for particular parts of the body, such as, the legs, knees or joints, since the wound care device here is usually configured in the form of sleeves.

Moreover, in a special embodiment, it is provided that the wound care device comprises in addition at least one nutrient, at least one disinfecting and/or decontaminating and/or at least one protease-inhibiting active ingredient and/or active ingredient complex.

The active ingredient and/or active ingredient complex with disinfectant action can be, for example, a composition consisting of at least one vitamin or vitamin derivative, at least one metal ion as well as at least one detergent. Similarly, it can be a BUS (bacteriocin like inhibitory substance) or a coated magnetic particle.

The active ingredient and/or active ingredient complex with nutrient action can be a composition containing at least the components of an enteral and/or parenteral dietary supplement. Similarly, it can be at least one active element selected from the group comprising insulin, recombinant insulin, proinsulin, an insulin-like growth factor (IGF), an insulin mimetic and/or a diabetic specific non-glucose or sucrose-based energy carrier.

The active ingredient and/or active ingredient complex with protease-inhibiting action can be at least one active element selected from the group containing protease inhibitors, superabsorbent polymers, chelators for bivalent cations, collagen, coated magnetic particles, acids, buffers, nonpathogenic acid-producing microorganisms, probiotics and/or symbiotics.

Additional connections and contexts pertaining to the nutrient, disinfecting or decontaminating and/or protease-inhibiting active ingredients and/or active ingredient complexes are described in DE102007030931 of the applicant of the present invention, to the entire content of which reference is made here. In DE102007030931, further nutrient, disinfecting or decontaminating and/or protease-inhibiting active ingredients and/or active ingredient complexes are also described, which are also considered disclosed in this application.

Moreover, the wound care device according to the invention can also be introduced under negative pressure into a wound supply system for wound drainage. Such systems are disclosed, for example, in the documents DE202004017052, WO2006048246 and DE202004018245 of the applicant of the present invention, the disclosure content of which is considered to be part of the present invention.

From the first invention, a device for wound treatment using a negative pressure is known, comprising a gas-proof wound covering element, which when applied on the body of the patient forms a space remaining between the respective wound and the wound covering element, and at least one connection site which is in contact with the space, and through which air located in the space can be discharged, wherein at least one flat wound care device which absorbs the wound secretions lies over the wound covering element, and the volume of said device increases during the course of the absorption process, so that the absorbed wound secretions remain within the wound care device and thus beneath the wound covering element until the removal of the wound care device from the body of the patient, the wound care device is at least one layer of a textile section which is enriched with superabsorbent substances, and which is surrounded by a liquid-permeable sheath, and the layer, in a top view of its flat side, has an area which is 3-90% smaller than that of the sheath, so that the cross section of the wound care device can approximate a circular shape when close to its total filling capacity.

From the second invention, a multicomponent dressing for treating wounds of the human or animal body is known which uses negative pressure, comprising: a wound covering element for the application on the skin and on a mucosal surface of at least one contact site which is in contact with the wound space, and through which the substances located in the wound space can be discharged, wherein this space comprises superabsorbent polymers, wherein the absorbed wound secretions remain bound to the polymers in the wound space until removal from the wound space, wherein the polymers as a result of their binding capacity support mutual synergies with the subatmospheric pressures.

From the last invention, a drainage device is known for the treatment of wounds using a negative pressure, comprising a gas-proof wound covering element consisting of a film-like material, which is attached adhesively when applied on the body of the patient to the skin surface around the wound area, and which forms a sealed space remaining between the respective wound and the wound covering element, at least one drainage tube which can be inserted into the space, through which the substances located in the space can be discharged, and at least one wound care device which absorbs the wound secretion by suction and is arranged within the space, and which comprises at least one layer of a textile section with a high content of superabsorbent substances, which is surrounded by a fluid-permeable sheath, wherein the absorbed wound secretions remain within the wound care device and thus beneath the wound covering element until the removal of the wound care device from the body of the patient, and wherein the wound covering element comprises a gas-proof closable treatment opening, through which the wound care device can be inserted into the space and removed from the space.

The wound care device according to the invention in addition has a shape adapted to the anatomical circumstances. For this purpose, it is possible, for example, for the wound care device to be configured in the shape of a sleeve which can be fitted over an arm or a leg or a joint, or it can be in the form of a dressing adapted to the heel, to the elbow joint or the like.

The wound care device according to the invention can, moreover, be configured in such a manner that it is suitable for being placed around a surgically positioned line. For this purpose, the wound care device can comprise, for example, at least one slit, which makes it possible to place the dressing on the body of a patient around a line (for example, a drainage line or a catheter), wherein a second, also flat, wound care device is associated with the former wound care device, the latter wound care device being spaced from the former wound care device, wherein the distance is bridged by a connection strip or bar. Such a wound care device is known, for example, from DE202006005966 of the applicant of the present invention, whose entire content is to be added to the disclosure content of the present description.

In this connection, it is also preferable to provide that the wound care device comprises at least one agent which can limit bleeding or the tendency to bleed. Said agent can be at least one chemically and/or physiologically active ingredient or active ingredient complex, or at least one physically active ingredient.

For this purpose, it is possible, for example, for the wound care device to be configured
substantially as a flat material section comprising an absorption material, which is formed from a suctioning nonwoven with superabsorbent polymers distributed therein as well as with at least one chemically and/or physiologically acting active ingredient or active ingredient complex,
in the form of or in combination with a pressure or compression bandage,
as a combination of a primary wound dressing material which does not absorb or absorbs only insignificantly, which comprises at least one chemically and/or physiologically acting active ingredient or active ingredient complex, and a secondary wound dressing material arranged peripherally with respect to said primary wound dressing material and containing superabsorbent polymers, wherein a diffusion barrier is optionally arranged between the two,
in the form of a small dressing packet, comprising a primary wound dressing with at least one chemically and/or physiologically acting active ingredient or active ingredient complex as well as with a packing section arranged on the wound dressing material, which comprises superabsorbent polymers at least in some sections, and/or
as a material section having a longitudinal extension comprising an absorption material, wherein the material section has resiliently deformable properties, and wherein the material section comprises superabsorbent polymers as well as optionally at least one chemically and/or physiologically acting active ingredient or active ingredient complex.

The chemically and/or physiologically active ingredient or active ingredient complex is preferably at least one substance or composition which has hemostatic properties. These substances are known under the general term “hemostatics.”

The active ingredient with physical activity is, for example, a tourniquet, a pressure pad, a pressure bandage or a compression bandage.

The present invention is explained in further detail in the figures shown and discussed below. It must be taken into consideration here that the figures only have descriptive character and that they are not intended to limit the invention in any way. The figures show:

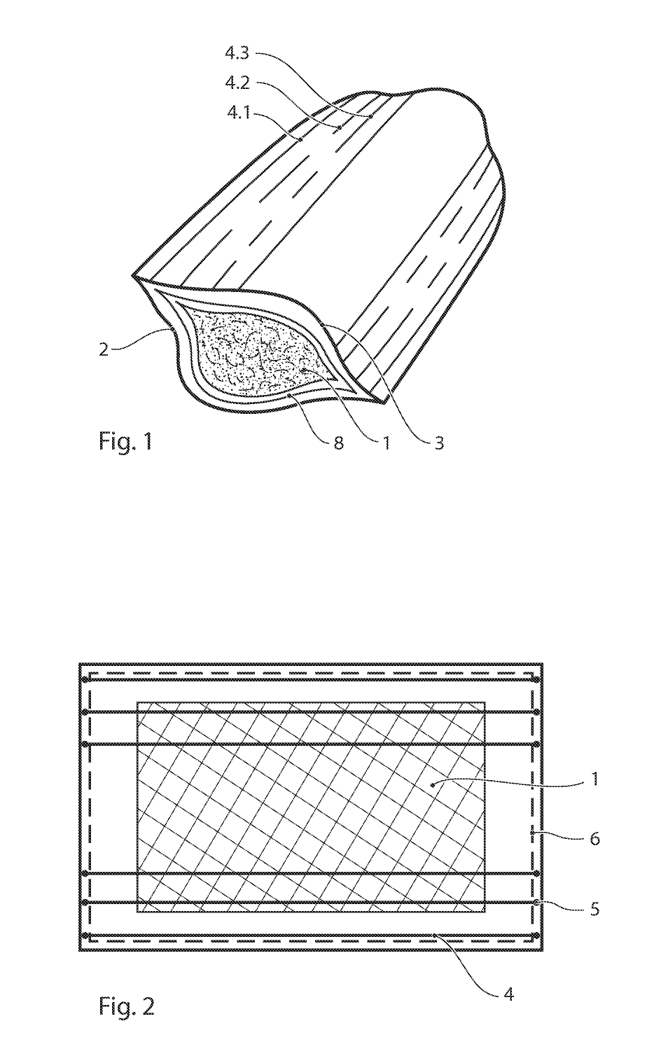

FIG. 1 shows a wound care device according to the invention with an absorbent body 1 in an inner, fluid-permeable sheath 8, and a target-oriented deformable outer sheath 2, 3 with a bottom side 2 which faces the wound, and an upper side 3 which faces away from the wound. Moreover, the sheath 2, 3 has 3 visibly incorporated threads 4.1, 4.2, 4.3. As a result of a volume increase of the absorbent body in the horizontal direction, which is caused by the fluid absorption, and which is indicated by the bulging in the upper area of the figure, the threads 4.2 and 4.3 have torn. The interruptions as such as well as their number thus give an indication of the extent of the volume increase of the absorbent body.

FIG. 2 shows a top view of the wound care device according to the invention. The absorbent body 1 is indicated by the crosshatched rectangle in the center, which is surrounded by a partially resilient sheath with a peripheral seam 6. The seam 6 can also be resilient in partial zones, for example, on the right and on the left, so that the sheath, when it is in use, can expand both in the vertical and also in the horizontal direction. On the outer side of the sheath which is turned away from the wound, 3 threads 4 are incorporated, which are connected at attachment points 5 to the sheath. In the case of an expansion of the absorbent body, the sheath located around said body also expands. In the process, an expansion in the vertical direction occurs, that is to say away from the wound, and in the horizontal direction, that is to say in the partial zones with resilient seams. With the increase in the volume of the absorbent body located beneath, the threads 4 tear, and thus give an indication of the extent of the absorption of wound exudate. Since, as represented here, threads 4 are incorporated in the sheath at different distances from the margin, they also tear at different times during the expansion. As a result, the care staff person/the patient receives an additional indication of the status of the absorption process.

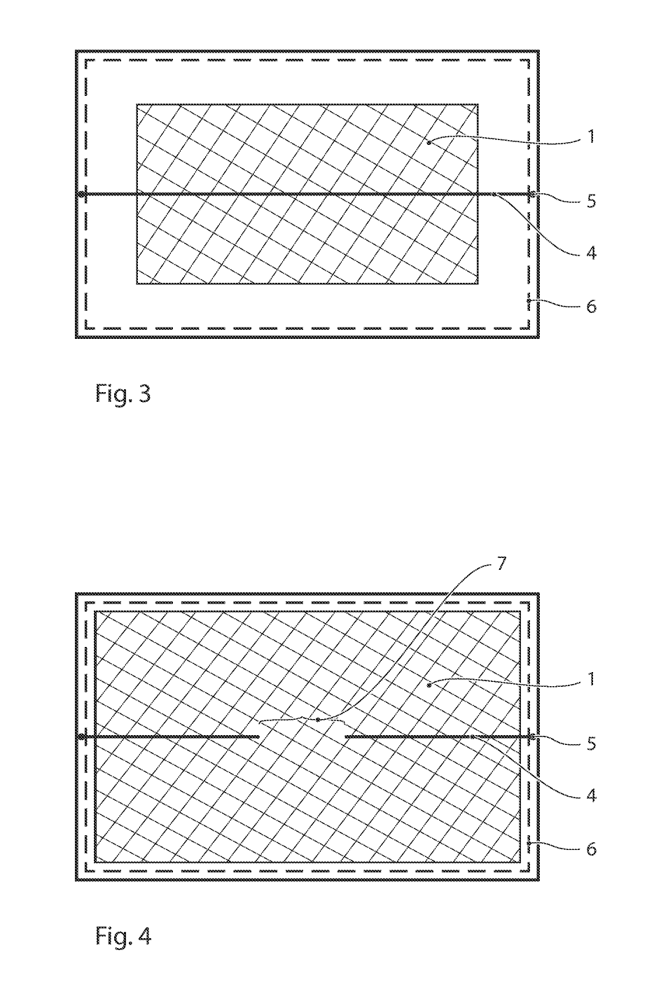

FIG. 3 also shows precisely a wound care device according to the invention having absorbent body 1 in an elastic sheath 1 with peripheral seam 6 and thread or wire 4 incorporated therein, with the associated attachment points on the two sides 5.

FIG. 4 shows the wound care device according to the invention, as already depicted in FIG. 3, but with the torn thread 4 and the distance 7 formed as a result between the two thread ends.

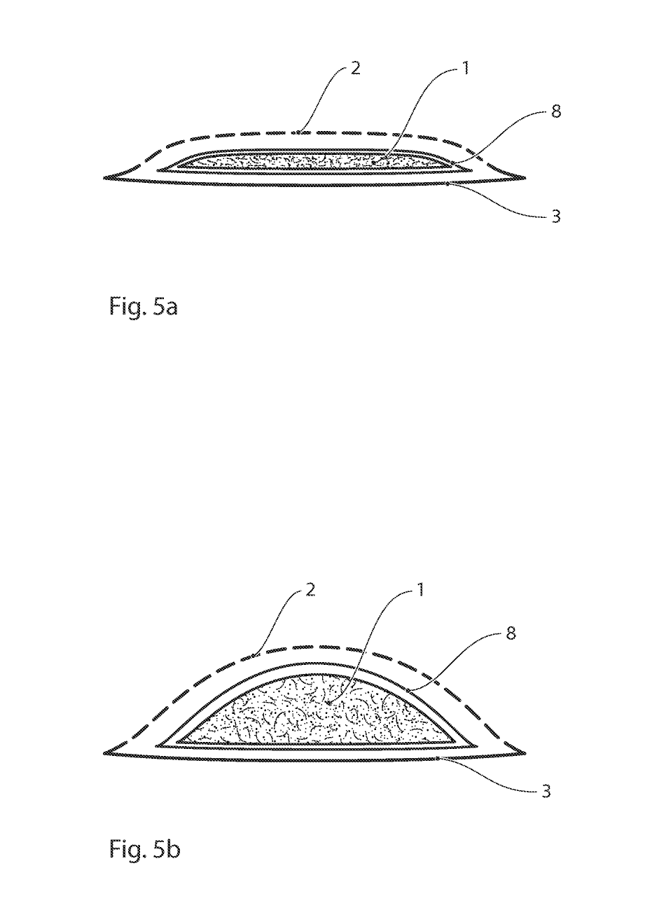

FIG. 5a shows the wound care device according to the invention in cross section from the side. The absorbent body 1 is surrounded by an inner fluid-permeable sheath 8 which adapts to the volume increase of the absorbent body 1. Around it, the target-oriented deformable sheath with a resilient top side 2 (which is shown by the broken line representation) and a nonresilient bottom side 3 (which is shown by the solid line representation) are located. The wound care device can be used with the top side either turned toward the wound or turned away from the wound, depending on the desired direction for the expansion of the absorbent body 1.

FIG. 5b shows the wound care device according to the invention as already represented in FIG. 5a, but after the absorption process. The sheath has adapted to the volume increase of the absorbent body 1, and it has expanded upward in a target-oriented manner.

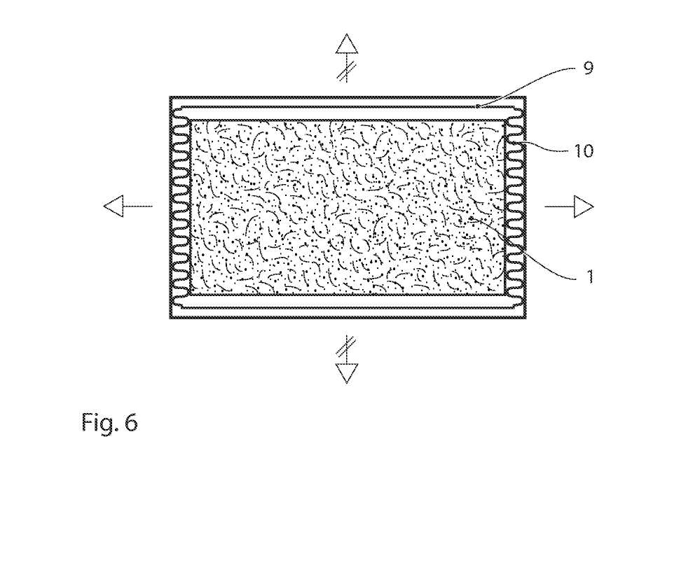

FIG. 6 shows the wound care device according to the invention with the absorbent body 1 and a seam in the periphery of the sheath, which is resilient 10 in partial zones and nonresilient 9 in other partial zones. For the elastic partial seams 10, it is possible to use a gathered ribbon with a proportion of rubber, for example. The target-oriented expansion of the wound care device is indicated by the arrows on the sides in which the sheath is held together by the elastic seams 10. Accordingly, no expansion occurs in the areas with nonresilient seams 9—represented by the solid-line arrows above and below the wound care device. A similar behavior can also be achieved with unidirectional resilient sheath material, that is a material which is expandable in one direction but not in the direction orthogonal to the former direction. Such materials are known from WO2005087159, for example, in which they are used in a joint bandage.

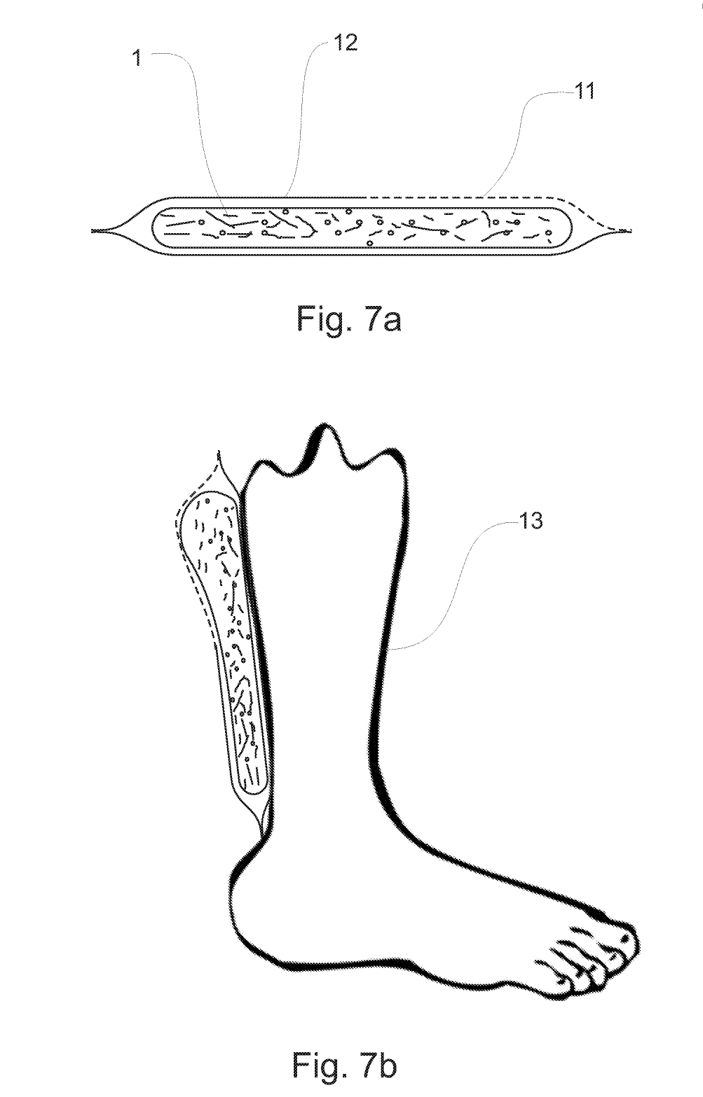

FIG. 7a shows an additional wound care device according to the invention in cross section from the side. The absorbent body 1 is surrounded by a liquid-permeable sheath, which has a resilient section 11 (which is shown by the broken-line representation) and a nonresilient section 12 (which is shown by the solid line representation).

FIG. 7b shows said wound care device in position on the injured leg 13 of a patient. As a result of the mentioned combination of resilient and nonresilient sheath materials, it is ensured that the liquid (exudate) absorbed by the absorbent body from the wound does not sag downward due to gravity into the lower area of the absorbent body and does not produce a sack-like formation there—which therefore can be unpleasant for the patient, since under some circumstances this sagging can be visible to third parties as a bulge on the leg of the pants of the patients, possibly interfering with the patient's walking, and potentially even bursting open.

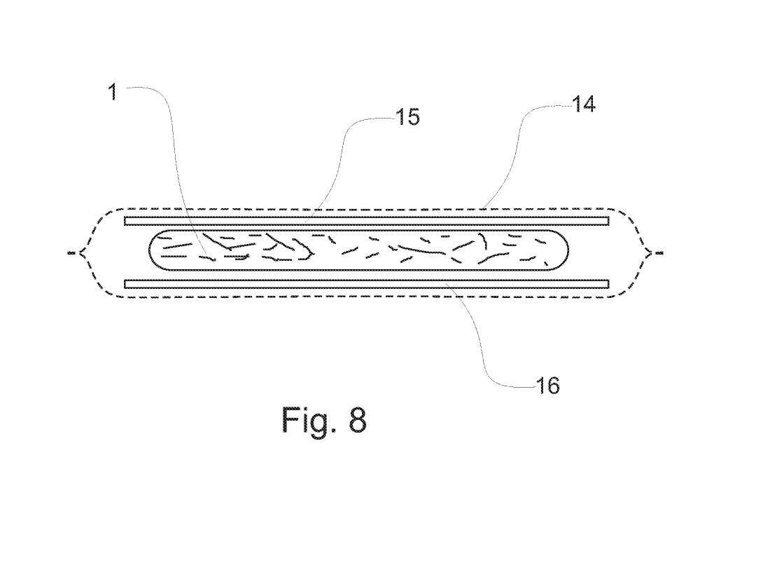

FIG. 8 shows an additional wound care device according to the invention in cross section from the side. The absorbent body 1 is surrounded by a liquid-permeable sheath 14, which, in the depicted example, consists entirely of a resilient material (which is shown by the broken line representation). The sheath is applied closely to the intermediate layers 15, 16, which are arranged on the two sides of the absorbent body 1, and which preferably consist of a nonresilient material, for example, of a cellulose nonwoven without superabsorbent polymers. The two intermediate layers protrude at least on two sides over the absorbent body, thus they laminate said body as it were and hide it from the outside, and in this manner define an expansion space which the absorbent body can use for its volume increase when it absorbs fluid. If the absorbent body has filled the space made available in this manner, the additional volume increase is defined by the expansion properties of the elastic sheath.

1. Wound care device having at least one absorbent body (1), which is surrounded by an outer sheath, characterized in that the sheath comprises means which are configured and/or selected in such a manner that the sheath is at least partially deformable in a target-oriented manner by a volume increase of the absorbent body (1) which is caused by the fluid absorption.

2. Wound care device according to claim 1, characterized in that the sheath delimits an expansion margin around the absorbent body, which is adapted in terms of its dimensions to the volume increase of the absorbent body, and which, in the starting state, is 3-90 volume % greater than the absorbent body.

3. Wound care device according to claim 2, characterized in that at least one lamination layer, preferably made of cellulose nonwoven, is arranged between the absorbent body and the sheath.

4. Wound care device according to claim 3, characterized in that the sheath comprises a fluid-permeable side (2) which is turned toward the wound, and a side (3) which is turned away from the wound, and at least one of the two sides is at least in partial zones resiliently and/or plastically deformable.

5. Wound care device according to claim 4, characterized in that the sheath is made of a largely nondeformable material and a resiliently and/or plastically deformable material.

6. Wound care device according to claim 5, characterized in that the deformable material is a fabric or nonwoven having at least one resilient or plastic component, preferably a chemical fiber, particularly preferably a polyaddition fiber, and most preferably a polyurethane fiber.

7. Wound care device according to claim 5, characterized in that the deformable material is a plastic film, preferably a polyethylene or polyurethane film.

8. Wound care device according to claim 7, characterized in that the sheath comprises on at least one side a peripheral seam (9, 10), preferably an ultrasound or welding seam, for delimiting the deformation of the sheath, which is preferably resiliently and/or plastically deformable at least in some sections, and which is in particular preferably gathered.

9. Wound care device according to claim 8, characterized in that the absorbent body (1) comprises rapid distribution layers for the absorption of wound exudates.

10. Wound care device according to claim 9, characterized in that the absorbent body (1) comprises partially superabsorbent substances in the form of particles, granulates, powders, bulk preparations, fibers, a preform, a foam, a fiber knitted fabric, a fiber roving fabric or a fiber nonwoven fabric and/or a fiber wadding.
